(19) United States
(12) Patent Application Publication
(10) Pub. No.: US 2008/0058787 A1
(43) Pub. Date: Mar. 6, 2008

(54) Methods, Devices, and Kits For Polymerization of Regenerative Biomaterials on Tissue Surfaces

(76) Inventor: Michael Gertner, Menlo Park, CA (US)

Correspondence Address: MICHAEL GERTNER, P.O. BOX P, MENLO PARK, CA, 94026, US

(21) Appl. No.: 11/735,492

(22) Filed: Apr. 16, 2007

Related U.S. Application Data
(60) Provisional application No. 60/745,092, filed on Apr. 18, 2006.
(60) Provisional application No. 60/807,611, filed on Jul. 17, 2006.

(51) Int. Cl. A61B 18/18; A61F 2/02

(52) U.S. Cl. 606/14; 606/13; 606/3; 623/11.11; 623/23.75

(57) ABSTRACT

A kit including a surgical instrument and a DC power pack, the surgical instrument adapted to create a change in a material applied to a body surface. In one embodiment, the body surface is an articular surface, the material is a hydrogel, and the surgical instrument carries an LED.

### BACKGROUND

Cartilage reconstruction has been the holy grail of restorative medicine. To date, arthroplasty (joint replacement) is the most widely accepted method of repairing irreparable cartilage damage. However, not all patients are candidates for this type of surgery, some being too young and others too old.

Carticel™ is an approved procedure for the less invasive repair of cartilage. The procedure required for Carticel™ is that patients receive an initial biopsy of their cartilage which is then shipped to a lab in Massachusetts, where the cells are incubated and expanded. Thereafter, the cells are sent back to the surgeon who reimplants them in the patient. This procedure is time consuming and expensive.

A further complication with this procedure is that the cell matrix in which the cells reside is not necessarily conducive to adhering to surrounding cartilage. Typically, surgeons will adhere a patch over the cartilagenous cells and will tack the patch to surrounding cartilage.

What is needed therefore is a simple, expedient method and material to repair cartilage and adhere the material to surrounding cartilage.

Recent work has focused on synthetic biomaterials to repair defects in cartilage. In some cases, these biomaterials are activated by energy sources such as light. One of the difficulties inherent in these techniques can be that the material is difficult to apply, hold, and stick prior to application of sufficient light to activate the material. Another complexity is that typical surgical light sources require an optical fiber to be draped across the surgical field as well as a base unit from the light is generated.

A light emitting diodes (LED) is generally a device which requires a DC current to generate light. LED refers to a light generating chip and a package surrounding the chip. The package around the chip supplies the necessary heat transfer as well as optical components to alter the light paths. The LED can contain one chip or several chips, and correspondingly, more than one optical component for each chip. Each chip receives its own current and can generate its own peak wavelength. Typically, a chip emits a peak wavelength with a 10-15 nm tail on either side.

### SUMMARY OF INVENTION

To address the above clinical needs, a method, a kit, and a surgical instrument (device) to polymerize a regenerative material (“material”) placed on a tissue surface is described; in some embodiments, the material is placed on a surface in a body cavity. In one embodiment, the invention comprises: inserting a device into a fluid fillable body cavity wherein the device comprises a light emitting diode (LED); the LED is powered by a DC power supply (e.g. a battery) such that the LED emits light of an intensity sufficient to polymerize the material in a time interval from 1 second to 20 minutes. In some embodiments, the LED emits light in the range 340 nm to 400 nm; in some embodiments, the LED emits light in the range from 250 nm to 360 nm. In some embodiments, the LED emits light in the range from 360-380 nm. In some embodiments, the LED emits light in the range from 400 nm to 700 nm; In some embodiments, the LED emits light in the range greater than 700 nm. In some embodiments, the total power of the light from the LED is from 0.1 mW to 50 mW when measured in a plane 0.5 cm from the LED and wherein the area of measurement is greater than 1 cm² but not greater than 5 cm². In some embodiments, the body cavity is a joint capsule and the joint capsule is filled with fluid. In some embodiments, the body cavity is an abdominal cavity. In some embodiments, the body cavity is a thoracic cavity. In some embodiments, the device further comprises a 1.5 Volt (V), a 3V, a 9V, or a 12V battery source. In some embodiments, the device further comprises a heat conducting element. In some embodiments, the heat conducting elements directs heat toward the outside of the body cavity. In some embodiments, the device is a component of a kit and the kit further comprises a sheath or a separate device with a lumen wherein the surgical instrument can slide in and out of the sheath while the sheath stays in place in the body cavity. In some embodiments, the sheath further comprises a valve to retain a fluid within the body cavity while the device is moving within the sheath. In some embodiments, the sheath or device with a lumen further comprises one, two or three lumens. In some embodiments, the sheath further comprises a compliant component at its distal end and in some embodiments, the compliant component is expandable; in other embodiments, the compliant component is expandable with fluid; in some embodiments the expandable component is a balloon. In some embodiments, the balloon forms a cap around the material or the surface in the body cavity. In some embodiments, the balloon transmits the light from the LED, the LED being place inside the balloon; In some embodiments, the compliant material is attached to the sheath. In some embodiments, the balloon comprises a lumen; in some embodiments, the device comprises a lumen and a dry gas supply is further attached to the device. In some embodiments, the gas is pushed through the lumen of the sheath to partially dry the region where the material will be placed prior to illumination of the material with the LED. In some embodiments, the device comprises a material lumen in which the material to be placed on the surface is pushed through the material lumen while the balloon cups the surface.

In a preferred embodiment, a method of polymerizing a material on a tissue surface of a body comprises: placing a sheath comprising a lumen and a compliant ring inside a joint capsule; advancing the sheath and lumen to the tissue surface; placing the compliant ring against the surface of the joint to create a watertight seal; applying a drying fluid or agent through the lumen of the sheath; applying a material through the lumen of the sheath and onto the surface of the joint; applying a surgical instrument (device) comprising an LED through the sheath; and illuminating the material on the surface of the joint to affect a change in the material. In some embodiments, the material change is polymerization; in some embodiments, the material change is cross-linkage with the body tissue surrounding the region where the regenerative material is placed. In some embodiments, a compliant ring the distal end of the sheath is fluid expandable; in other embodiments, the compliant ring is a balloon; in other embodiments, the compliant ring is deformable; in other embodiments, the compliant ring is a hydrogel; in other embodiments, the compliant ring can transmit light; in other embodiments, the compliant ring can be fluid expandable. In one preferred embodiment, the compliant ring can define a watertight region when the compliant ring is pushed into the surface of the body tissue.

### DESCRIPTION OF FIGURES

FIG. 1 depicts the surgical instrument in place inside a fluid fillable body cavity.

FIG. 2 depicts a distal portion of the surgical instrument.

FIG. 3 depicts an embodiment of the surgical instrument.

FIG. 4 depicts a functional embodiment of a distal portion of the surgical instrument.

FIG. 5 depicts a flow chart for the method of using the surgical instrument on a body surface.

FIG. 6 depicts a flow chart for a surgical instrument inside a joint cavity.

### PRIORITY DATA

This application claims priority to the following applications:

Ser. Nos: □az60/745,092 and 60/807,611.

### INCORPORATION BY REFERENCE

Patent Application Nos:
20070048291
20050196377
20050059572
20040170663
20050130324
U.S. Pat. No. 6,224,893

### DESCRIPTION OF INVENTION

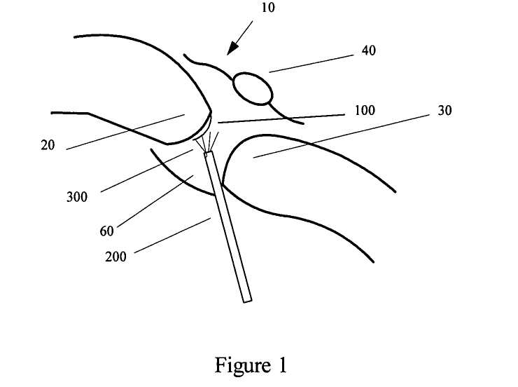

FIG. 1 depicts an example of a joint 10 which in one embodiment, is a knee joint. Other joints include facet joints, hip joints, joints of the hand, elbow joints, shoulder joints. In other embodiments, other internal organs or body cavities or tissue surfaces are accessed. Examples include bony vertebrae, intervertebral discs, intra-abdominal or intrathoracic organs such as the liver, spleen, stomach, kidneys, ureters, small bowel, large bowel, esophagus, gastro-esophageal junction, heart, and lungs. Other tissues include soft tissues such as muscle, skin, cartilages (e.g. nose, ear, intracostal cartilage).

In FIG. 1, the femur 20, the tibia 30, and the patella 40 are depicted. The inside of the joint, the capsule, is also depicted 60. A material 100 is depicted on one cartilage surface, in this example, the femoral condyle 20. The material 100 can be placed on the surface of the joint to induce cartilage regeneration or prevent bone on bone rubbing which causes pain.

Material 100 is placed on the cartilage and in some cases covers a defect on the surface of the cartilage. Material 100 can be responsive to electromagnetic energy. In some embodiments, the electromagnetic energy is current which oscillates at a frequency in the radiofrequency portion of the spectrum. In other embodiments, the energy is in the microwave part of the spectrum and in other embodiments, the energy is in the infrared portion of the spectrum. Preferably, the material 100 is responsive to electromagnetic radiation in the visible or ultraviolet wavelengths of the electromagnetic spectrum.

“Responsive” refers to a property of the material which is changeable. For example, in some embodiments, when radiation or electromagnetic energy is applied to the material 100, the material 100 can be cross-linked to from a structure. Alternatively, the material 100 can polymerize in addition to or in place of cross-linking. The material 100 can also be induced to aggregate, swell, heat, cool, gel, dry, wet, and desolvate (evaporate a solvent).

Material 100 in some embodiments is a material responsive to light (e.g. see the following U.S. patents and patent applications all of which are incorporated by reference: U.S. Pat. No. 6,224,893, 20050069572, 20040170663, 20050196377) such as ultraviolet light.

In other embodiments, the material is a material responsive to infrared light. In some embodiments, the material has a nanomaterial component such as carbon nanotubes, nanoparticles (e.g. metallic nanoparticles, polymer nanoparticles, ceramic nanoparticles or combinations therein), nanowires (e.g. silicon, carbon, carbohydrate, protein, DNA, etc.), nanoshell (e.g. see patent no. 20050130324 incorporated by reference in its entirety). These materials can further have organic molecules attached which then facilitate cross-linking of the particles to one another to create a material. The organic molecules can be directly responsive to energy or they can be indirectly responsive to energy via the nanomaterials which themselves can absorb the energy.

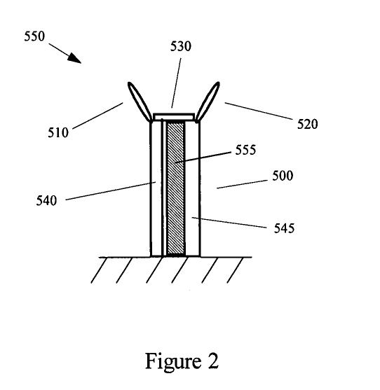

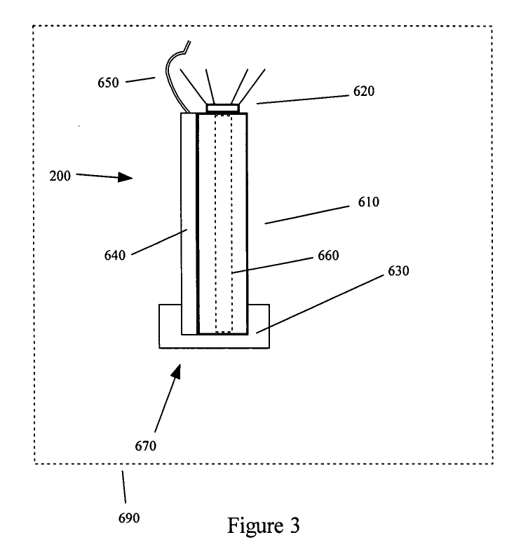

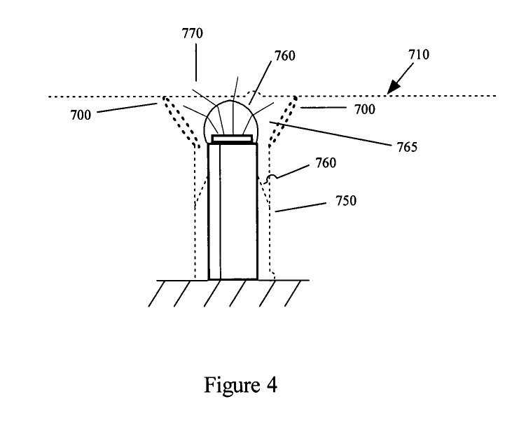

Surgical instrument (device) 200 has a distal end 550 (FIG. 2) and a proximal end 670 (FIG. 3) and can emit radiation (e.g. UV radiation) from the proximal end 670 or distal end 550 toward material 100. When light is transmitted from the proximal end 670, light is transported longitudinally through the device (e.g. through a light guide or fiber bundle and out the distal end 550) to the material. Light (or other energy source) can also originate at the distal end. When light is emitted from the distal end 550, light does not have to be transported because it is at the point of use. In some embodiments, a protective coating, light conditioner, or lens 760 covers the device (FIG. 4). In other embodiments, the light conditioner or lens 760 is adjustable. For example, lens 760 can have an adjustable focal length which can alter the spot size created by the light (in the case light is the energy source applied to the tissue). Device 200 can further have an adjustable current source which increases or decreases the power to the energy source and therefore increases or decreases the energy incident on the material 100.

Device 200 can further contain sensing devices or instrumentation. Such devices can include CMOS or CCD elements such that tissue can be visualized as energy is applied. Furthermore, optical spectroscopy methods can be used in which light is applied to the tissue or the material and the reflected light analyzed to monitor a reaction occurring in the tissue or in the material 100.

Radiation 300 (FIG. 1) can be one or more of: ultraviolet, visible, infrared, microwave, radiowaves, or current. Radiation can require direct contact such as in the case of electromagnetic energy transferred to the material 100 by a radiofrequency electrosurgery device. In preferred embodiments, however, radiation is delivered via non-contact methods (e.g. light). In one particularly preferred embodiment, the light source is an LED source such as an LED source. In some embodiments, the LED source emits UV light with one or more peaks in the spectral range from 250 nm to 400 nm. In some embodiments, the LEDs emit light with one or more peaks in the visible spectral range from 401 nm to 750 nm. In further embodiments, the LEDs emit light with one or more peaks in the infrared portion of the electromagnetic spectrum (751 nm-10 microns). In some embodiments, energy source is a coherent light source such as a laser.

Importantly, surgical instrument 200 can be powered using a DC source such as a battery. The battery can in some embodiments be 1.5V, 3.0V, 9.0V, or 12 V. The importance of these power sources is that they are direct current (DC) sources and therefore are inexpensive and disposable. They are also sterilizeable via autoclave, ethylene oxide, gamma radiation, and the like. In some embodiments, the DC power source can be directly (e.g. rigidly) coupled to device 200; in other embodiments, DC power supply is connected by wires to the device. In some embodiments, surgical instrument 200 is powered by an AC power source which is transformed into a DC source. In this embodiment, the power supply can be rechargeable.

Surgical instrument 200 can be supplied in a sterilized package to the surgeon. Sterilization can be performed with or without the power supply as part of the instrument. Typical forms of sterilization include ETO, autoclave, gamma radiation, hydrogen peroxide, carbon dioxide, and electron beam.

FIG. 2 depicts distal end 550 of device 500. In some embodiments, a first compartment 545 is included in the device 500. In some embodiments, a second compartment 540 is included in the device 500. Either or both compartments can be lumens. Either lumen can be used to transfer materials, fluids, or substances to the body cavity. Compartments 540 and 545 can also be used to transfer heat and/or electricity to the inside of the body cavity in some embodiments. In some embodiments, a heat transfer tube (e.g. a heat pipe) 555 is supplied within compartment 540 or 545 which would allow heat to be transferred efficiently to or from a tissue surface.

In other embodiments, device 500 further comprises structures 510 and 520. These structures 510 and 520 can be fluid expandable; in other embodiments, structures 510 and 520 are compliant but not necessarily expandable; for example, they can contain a gel such as a hydrogel. In some embodiments, the material is an elastomer. In some embodiments, 510 and 520 are the same structure and represent a continuous ring. In one embodiment where 510 and 520 are the same structure, the structure is a fillable balloon or a balloon which is supplied filled with a material but not fillable per se. The balloon can have lumens and/or perforations and/or can be transparent or can conduct electromagnetic energy (e.g. transparent to ultraviolet light or can conduct electrical energy). In some embodiments, one of the compartments 540, 545 communicates with the expandable structure(s) and/or balloons 510, 520.

Radiation emitting device 530 can be positioned on top of either compartment or can be placed on an intermediate mount (such as an LED submount which is well known in the art) between the compartment and the radiation emitting device 530. Radiation emitting device 530 can communicate with the proximal end of the device 500 through the compartments 540, 545. For example, electrical power and/or heat conduction can take place through the compartments and can aid in the functioning of the radiation emitting device.

Device 200 can have many features considered desirable to a surgeon. It can be powered by a portable battery pack 630 (FIG. 3). Battery pack 630 can be attached to the outer portion of the device 610. Battery pack can be a rechargeable battery or a battery such as an alkaline, lithium ion, or nickel cadmium. Device 200 can further be sterilizeable or disposable. The battery pack can be sterilizable and disposable as well. For example, the battery pack can include batteries such as a 9V battery which is portable on the surgical device, sterilizeable on the surgical device, and disposable with the surgical device.

An injection port 640 can also be included on the device 610 in some embodiments; in other embodiments, injection port 640 communicates with a flow director 650. Structure 620 can be a radiation emitting structure such as an LED. Structure 620 can include a heat transferring element which disperses the heat generated by the LEDs. Structure 620 can further communicate with a heat conducting rod 660 which also can transfer heat away from the distal end of device 610. Device 610 and its components can be packaged in their entirety into a sterilized package 690 for delivery to the surgeon; such a package can be a completely disposable one in which the device is thrown away after the procedure is finished.

FIG. 4 depicts a sheath 750 through which surgical instrument 200 is slideable. Sheath 750 can be a standard port type device used in minimally invasive surgery or can be a specialized device created for the surgical instrument of this invention, the specialized device then slideable through a typical port for minimally invasive surgery. Sheath 750 has a lumen and can contain a valve 760 through which the device 200 slides; the valve prevents fluid from leaving the tissue surface or the body cavity through valve 750. Sheath 750 contains compliant structures 700 which enable the sheath to be pressed against the body surface 710 to create a seal, preventing liquid from entering the sealed region 765 within compliant structures 700 discussed above. Sheath 750 can be pressed against the body surface 710 to create the seal and materials can be placed in and out of the sheath without losing them outside the region 765. Gases such as air, carbon dioxide, or nitrogen can be used to at least partially dry body surface 710 prior to a responsive or polymerizeable material 100 being applied to the body surface. After material 100 is placed, then device 200 can be placed through the sheath 750 to illuminate and polymerize/cross-link the responsive material 100. Device 200 in FIG. 4 further comprises a structure 760 which conditions the light 770 as it passes through. The conditioner 760 can be a lens, a diffuser, or a focusing element and can be made out of moldable or machineable materials. Alternatively, despite its name, structure 760 does not condition light at all but merely provides for protection of the LED structures.

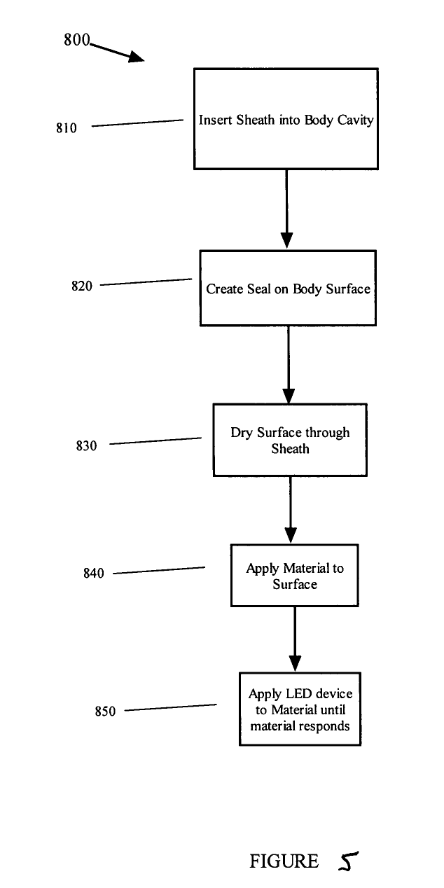

FIG. 5 depicts a method 800 of applying a material to a body surface in a body cavity. Steps include: a) Insertion of a sheath into the body cavity 810; 2) Creating a seal 820 on the body surface with or without the sheath; 3) drying 830 the surface through the seal created by the sheath; 4) Applying 840 the material through the sheath and to the body surface; 5) Applying 850 radiation from an energy emitting device (e.g. an LED device) to the material for the appropriate amount of time to cause cross-linking and/or polymerization.

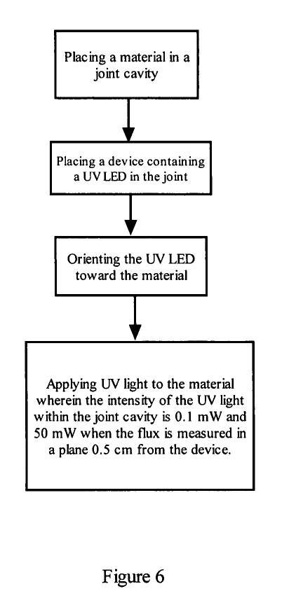

FIG. 6 depicts another method 900 of applying a material to a body surface in a body cavity. In this method, a photo-curable material is placed 900 in a joint cavity; a device containing at least one UV LED is placed 910 in the joint capsule; the device is oriented so as to illuminate the material with UV light 920; the method further comprises applying UV light to the material wherein the intensity of the UV light within the joint cavity is between 0.1 and 50 mW when the flux is measured in a plane 0.3 to 5 cm from the LED.

1. A method to polymerize a polymer inside a body of a subject comprising:
removing a sterile surgical instrument from a sterile container, said surgical instrument comprising at least one LED which can emit at least one wavelength;
placing the surgical instrument comprising at least one LED emitting at least one wavelength inside the body of the subject;
activating said LED through a switch associated with the probe said activating step leading to a current draw from a DC power source;

2. The method of claim 1 wherein said surgical instrument is adapted to enter a fluid fillable body cavity through a percutaneously placed access port.

3. The method of claim 2 wherein said body cavity is a joint cavity.

4. The method of claim 3 further comprising the step of inserting a device with a lumen said device with a lumen adapted to receive said surgical instrument comprising a distal end wherein said distal end is further adapted to enable a fluid tight seal with a tissue inside said joint cavity.

5. The method of claim 4 further comprising: applying a gas through said lumen to said tissue to at least partially dry said tissue; placing a polymer through said lumen to contact said tissue; activating polymerization of said polymer through emission of light from said LED of said surgical instrument.

6. The method of claim 5 wherein said LED is an ultraviolet LED with a peak wavelength between 250 nm and 350 nm.

7. The method of claim 5 wherein said LED comprises at least one ultraviolet emitter with a peak wavelength between 250 nm and 400 nm.

8. The method of claim 5 wherein said LED comprises at least one ultraviolet emitter with a peak wavelength between 250 nm and 400 nm and at least one second emitter with a wavelength greater than 400 nm.

9. The method of claim 7 wherein said ultraviolet emitter emits between 0.5 mW and 5 mW over an area of 1 mm² to 1 cm².

10. A kit to polymerize a polymer inside a body of a subject comprising:
a sterile surgical instrument comprising at least one LED adapted to emit at least one wavelength of light;
a direct current battery pack adapted to power the LED.

11. The kit of claim 10 further comprising a polymer activateable by light from the LED.

12. The kit of claim 10 wherein said sterile surgical instrument further comprises a protective layer for the LED wherein the protective layer covers the LED and is transparent to the light emitted from the LED.

13. The kit of claim 10 wherein said battery pack is supplied sterile.

14. The kit of claim 10 wherein said surgical instrument is adapted to fit through a percutaneous access port.

15. The kit of claim 10 wherein said surgical instrument is adapted to fit through a percutaneous access port, said access port sized to fit into a fluid fillable body cavity.

16. The kit of claim wherein said fluid fillable body cavity is a joint.

17. The kit of claim 15 wherein said fluid fillable body cavity is an abdomen.

18. The kit of claim 10 wherein said at least one wavelength of light is ultraviolet light.

19. The kit of claim 18 wherein said ultraviolet light is <365 nm.

20. The kit of claim 18 wherein said ultraviolet light is <400 nm.

21. The kit of claim 10 wherein said at least one wavelength of light is blue light between 400 and 450 nm.

22. The kit of claim 10 wherein said at least one wavelength of light is greater than 450 nm.

23. The kit of claim 10 further comprising a device with a lumen through which said sterile surgical instrument can slide in and out of.

24. The kit of claim 23 wherein the distal end of said lumen is configured to create a substantially fluid tight seal with a tissue.
